(19) United States
(12) Patent Application Publication
(10) Pub. No.: US 2002/0138089 A1
(43) Pub. Date: Sep. 26, 2002

(54) Suction ring for microkeratomes

(75) Inventors: A. Ramon Gutierrez Ortega, Valencia (ES); Vicente Castello Pico, Valencia (ES); Emilio Sanz Amoros, Valencia (ES); Jorge Sabate Calatayud, Valencia (ES); Salvador Santos Pacheco, Valencia (ES); Felix Santatecla Carro, Valencia (ES); Emilio Vilaseca Buitrago, Valencia (ES); Javier Mares Anton, Valencia (ES); Jose Rubira Fernandez, Valencia (ES)

Correspondence Address: John Murray, Ph.D., Brinks Hofer Gilson & Lione, NBC Tower, Suite 3600, P.O. Box 10395, Chicago, IL, 60610, US

(73) Assignee: Novosalud, S.L.

(21) Appl. No.: 10/101,894

(22) Filed: Mar. 20, 2002

(30) Foreign Application Priority Data
Mar. 21, 2001 (ES) 200100652

(51) Int. Cl. A61F 9/00

(52) U.S. Cl. 606/166

(57) ABSTRACT

This suction ring is composed of a ring-shaped body (1) with an incarcerated, bevelled front opening (2), with an anterolateral cannula (4) for connection to a vacuum pump and a dovetail guide (7) on which the microkeratome cutting blade moves. The lateral wall of the body (1) has a tapered truncated conical configuration, diverging backwards for adaptation to the eyeball, in order to increase the suction capacity to the eye. The front area has a wider stepped perimeter (6), which together with a bulge (8) on the outer edge and corresponding to the tips of the guide (7), increases the effective length of the latter, allowing better fixation of the cutting blade.

### OBJECT

The present invention refers to a suction ring which uses the depression generated by a vacuum pump connected through a flexible pipe to fix itself to the eyeball and thereby function as the support and guide for the microkeratome cutting blade.

The object of the invention is to improve the ring's capacity to stick to the eyeball and improve the connection of the cutting blade to the suction ring itself.

### BACKGROUND

The entity applying in this case is actually the holder of the Invention Patent with Application No. 9801787 which describes a microkeratome which, among other elements that are not presently of interest, includes a suction ring with a cylindrical outer wall, with a partially incarcerated and bevelled upper opening, with a frontal and lateral cannula for connection, after being fitted to the eyeball, to the vacuum source that will ensure that the said ring may remain perfectly stable on the eyeball while the cutting blade works upon the cornea.

Furthermore, the aforementioned ring also has a dovetail guide at the front, for the longitudinal displacement of the cutting blade whose base is configured according to the said guide.

Moreover, this is the usual configuration of all known suction rings for microkeratomes to date.

Obviously, the extent to which the suction ring is attached to the eyeball depends on the extent to which both elements, ring and eyeball, are adapted to each other. Similarly, the stability of the cutting blade on the said ring also depends on the connection length between the tracks of the suction ring and the cutting blade.

### DESCRIPTION

The suction ring proposed in the present invention has been conceived and structured with a view to enhancing the two aforementioned aspects, i.e. to improving the ring's capacity to attach itself to the eyeball and to improving the connection between the cutting blade and the dovetail on the suction ring.

To this end, in practice and according to the characteristics of the invention, it has been decided that the side wall of the ring, instead of the classical cylindrical configuration, should have a truncated conical shape, diverging towards the side that is to be attached to the eyeball. This means there is a greater surface of the aforementioned ring in contact with the eyeball and therefore a higher level of fixation between these elements.

In accordance with another of the characteristics of the invention, it has been decided that the ring shall include, on its front edge, a perimeter expansion that will increase the surface of the front side. At the same time, the perimeter of the front and the protruding side shall bulge over the tips of the grooves or scores of the dovetail guide. This shall in turn mean that the magnitude of the sectors of the guides, i.e. the sectors of the ring and the blade that are interconnected at any given time, shall also be widened. Therefore the stability of the cutting blade on the suction ring will be substantially enhanced.

### DESCRIPTION OF THE DRAWINGS

In order to complement the description that is being made and with the aim of allowing a better understanding of the characteristics of the invention, according to an example of the preferred embodiment of same, we include a set of drawings of an illustrative and non-restrictive nature, representing the following:

FIG. No. 1.- Shows a perspective view of a traditional suction ring for microkeratomes.

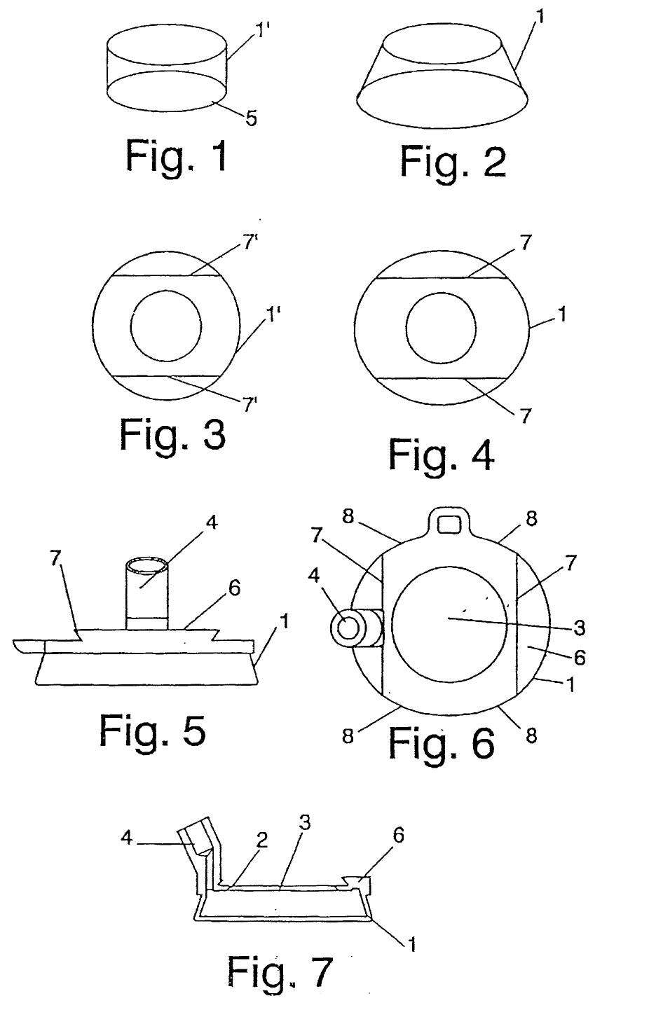

FIG. No. 2.- Shows, in a similar view to FIG. 1, a ring made in accordance with the object of the present invention, exaggerating its shape so as to allow the reader to contrast clearly and visually the effects achieved by the aforementioned shape.

FIG. No. 3.- Shows a ground view of a traditional ring.

FIG. No. 4.- Shows a similar view as in the previous figure, but of the ring that is the object of the present invention, also exaggerating its shape in order to show clearly the effects achieved by same.

FIG. No. 5. - Shows a side elevation of a specific practical embodiment of a suction ring for microkeratomes in accordance with the invention.

FIG. No. 6. - Shows a ground view of the previous figure.

FIG. No. 7.- Shows, finally, a side elevation and diameter section of the ring shown in FIG. 5, from a viewpoint that is displaced 900 with respect to the aforementioned figure.

### PREFERRED EMBODIMENT

In view of the figures described and particularly of FIGS. 5 to 7, we can see that the suction ring proposed by the present invention is composed, as is the case with any conventional ring, of a pipe (1) whose front opening (2) is noticeably incarcerated, with an inner bevel (3) allowing it to be attached to the front of the eyeball, whereas this pipe (1) has a cannula (4) to connect it, by means of a flexible tube, to a vacuum pump.

As regards the invention itself, the side wall of the body (1) has a truncated conical configuration, noticeably diverging towards the posterior edge of the ring, which is to be attached to the eyeball, as we can see in FIG. No. 7. Therefore, as it is obvious if we compare FIGS. 1 and 2, while a conventional ring (1′) has a trimming (5) that rests on the eyeball, the ring (1) that is presently proposed rests on the eyeball by means of an edge immediately adjacent to the aforementioned posterior edge. As we have explained before, and as it is obvious, this improves its adaptation and its capacity to attach itself to the eyeball.

Furthermore, it has also been decided that the body (1) shall include on its front edge, which corresponds to the incarcerated outlet (2), a perimeter stepped expansion (6), that increases the exterior perimeter of the frontal base of the body (1) and which, consequently, increases the length of the classical dovetail guide (7) on which the cutting blade of the microkeratome moves, as represented in the drawings.

Also with the aim of enhancing the length of the dovetail guide (7), it has been decided that the exterior perimeter of the front base of the body or ring (1), instead of adopting the traditional circular configuration shown in FIG. 3, shall present exterior bulges (8) corresponding to the tips of the aforementioned guide (7), which determine an irregular configuration for this perimeter, which also widens the said guides (7), as emerges from the schematic exploded representation in FIG. 4.

Therefore, given that the length of the tracks connecting both the suction ring and the cutting blade is increased, it is possible to achieve the objectives of the invention, i.e. on the one hand, to improve the attachment of the suction ring to the eyeball and on the other, to improve the working conditions of the cutting blade on the ring, and consequently, on the eyeball.

We claim:

1. Suction ring for microkeratomes, of the type composed of a ring-shaped body, with a noticeably incarcerated front opening that is attached to the eyeball, with an anterolateral cannula for connection by means of a flexible tube to a vacuum pump and with a dovetail guide on the front, along which the cutting blade moves, characterised by the fact that the side of the body (1) adopts a truncated conical configuration diverging towards the posterior area which is attached to the eyeball, in order to enhance the contact surface with the latter in the aforementioned posterior area.

2. Suction ring for microkeratomes, according to claim no. 1, characterised by the fact that the body (1) has a stepped expansion (6) on its front edge or area, meaning an increase in its outer perimeter which in turn means the dovetail guide is widened (7).

3. Suction ring for microkeratomes, according to the previous claims, characterised by the fact that the body (1), presents on the front area, corresponding to the perimeter stepped expansion (6), exterior bulges (8) corresponding to the tips of the guide (7), which mean complementary widening of the effective length of the aforementioned guide.
